(19) United States
(12) Patent Application Publication
(10) Pub. No.: US 2018/0228718 A1
(43) Pub. Date: Aug. 16, 2018

(54) COSMETIC COMPOSITION COMPRISING A CATIONIC SURFACTANT, A FATTY ALCOHOL AND TWO NONIONIC POLYMERS

(71) Applicant: L'OREAL, Paris (FR)

(72) Inventors: Ly-Lan NGUYEN, L'hay Les Roses (FR); Emilie SURAULT, Boulogne Billancourt (FR)

(21) Appl. No.: 15/749,881

(22) PCT Filed: Aug. 3, 2016
(86) PCT No.: PCT/EP2016/068532

§ 371 Date: Feb. 2, 2018

(30) Foreign Application Priority Data
Aug. 3, 2015 (FR) 1557455

(51) Int. Cl. A61K 8/898; A61K 8/34; A61K 8/41; A61K 8/73; A61K 8/87; A61Q 5/12

(52) U.S. Cl. CPC A61K 8/898; A61K 8/342; A61K 8/416; A61K 2800/594; A61K 8/87; A61Q 5/12; A61K 2800/48; A61K 8/731

(57) ABSTRACT

Cosmetic composition comprising a cationic surfactant, a fatty alcohol and two nonionic polymers The invention relates to a composition for the cosmetic treatment of keratin materials, in particular human keratin fibres such as the hair, comprising one or more cationic surfactants, one or more fatty alcohols, one or more nonionic non-associative cellulose-based polymers and one or more nonionic associative polymers. The invention also relates to a cosmetic treatment process using this composition.

Cosmetic composition comprising a cationic surfactant, a fatty alcohol and two nonionic polymers

The present invention relates to a composition comprising a combination of at least one cationic surfactant, at least one fatty alcohol, at least one nonionic non-associative cellulose-based polymer and at least one nonionic associative polymer, to the use thereof for the cosmetic treatment of keratin materials and to a cosmetic treatment process using this composition.

In the field of the cosmetic treatment of keratin fibres, especially human keratin fibres and better still of the hair, it is desired to care for keratin fibres subjected to various external attacking factors. Specifically, these fibres may be subjected to attacks of various origins, such as mechanical attacks, for example linked to disentangling or blow-drying, or else chemical attacks, for example following dyeing or permanent-waving.

These attacks have consequences on the qualities of the keratin fibre and may lead to difficult disentangling at the time of washing the hair, on dry hair and/or wet hair, and also to degradation of the surface properties of the fibres, which become rough and irregular at the surface, more particularly when the hair is dry.

Care products exist which make it possible to limit these phenomena. These products generally contain one or more agents for conditioning keratin fibres, such as cationic surfactants, fatty alcohols, silicones, etc. However, some of these conditioning agents such as fatty alcohols, which also make it possible to thicken the texture of the hair care products and to obtain a good “on surface” effect upon application, limit the depositing of the conditioning agents on the hair.

This drawback is especially observed with the combination described in European patent application no. 1088543, comprising a fatty alcohol, a cationic surfactant and at least one associative polymer.

There is therefore a need to develop compositions for the cosmetic treatment of keratin materials, which make it possible to overcome the above-mentioned drawbacks and to obtain good usage qualities such as easy and uniform distribution, an “on surface” effect upon application (that is to say a creamy and rich-feeling texture which coats the hair well) while retaining very good cosmetic properties due especially to an optimization of the depositing, on the hair, of the conditioning agents present in the formulation, especially fatty alcohols and cationic surfactants.

The applicant has now discovered that the combination of at least one cationic surfactant, at least one fatty alcohol and a specific thickening system comprising at least two polymers of specific nature made it possible to achieve the aims set out above.

A subject of the invention is therefore a cosmetic composition, comprising one or more cationic surfactants, one or more fatty alcohols, one or more nonionic non-associative cellulose-based polymers, and one or more nonionic associative polymers.

This composition has improved usage qualities such as easy and uniform distribution and an “on surface” effect upon application, while still providing improved depositing, on the hair, of the conditioning agents present in the formulation, especially fatty alcohols, cationic surfactants and optionally silicones, where present. In particular, the compositions according to the invention provide very good levels of performance in terms of disentangling, suppleness and smoothing.

Another subject of the present invention is also a process for cosmetic treatment, and more particularly for conditioning keratin materials, in particular human keratin fibres, such as the hair, comprising the use of the composition according to the invention.

Another subject of the invention consists of the use of a composition according to the invention for the cosmetic treatment of keratin materials, in particular human keratin fibres, and more particularly the hair.

Other subjects, characteristics, aspects and advantages of the invention will become even more clearly apparent on reading the description and the examples which follow.

In the text hereinbelow, unless otherwise indicated, the limits of a range of values are included in that range, especially in the expressions “between” and “ranging from . . . to . . . ”.

The expression “at least one” used in the present description is equivalent to the expression “one or more”.

According to the invention, the cosmetic composition comprises:
(a) one or more cationic surfactants,
(b) one or more fatty alcohols,
(c) one or more nonionic non-associative cellulose-based polymers, and
(d) one or more nonionic associative polymers,
   the weight ratio between the total amount of nonionic non-associative cellulose-based polymer(s) and that of nonionic associative polymer(s) ranging from 1 to 20.

The cationic surfactant(s) which can be used in the composition according to the invention comprise in particular optionally polyoxyalkylenated primary, secondary or tertiary fatty amines, and/or the salts or quaternary ammonium salts thereof, and mixtures thereof.

Mention may especially be made, as quaternary ammonium salts, for example, of:
those corresponding to the following general formula (I):

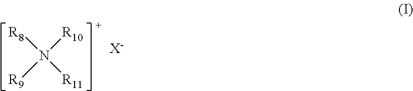

in which the groups R₈ to R₁₁, which may be identical or different, represent a linear or branched aliphatic group comprising from 1 to 30 carbon atoms, or an aromatic group such as aryl or alkylaryl, at least one of the groups R₈ to R₁₁ comprising from 8 to 30 carbon atoms and preferably from 12 to 24 carbon atoms; and X⁻ is an anion chosen from the group of halides such as chloride, bromide and iodide, phosphates, acetates, lactates, (C₁-C₄)alkyl sulfates, and (C₁-C₄)alkyl- or (C₁-C₄)alkylarylsulfonates.

The aliphatic groups may comprise heteroatoms such as, especially, oxygen, nitrogen, sulfur and halogens.

The aliphatic groups are chosen, for example, from C₁-C₃₀ alkyl, C₁-C₃₀ alkoxy, polyoxy(C₂-C₆)alkylene, C₁-C₃₀ alkylamide, (C₁₂-C₂₂)alkylamido(C₂-C₆)alkyl, (C₁₂-C₂₂)alkyl acetate and C₁-C₃₀ hydroxyalkyl groups; X⁻ is an anion chosen from the group of halides, phosphates, acetates, lactates, (C₁-C₄)alkyl sulfates, and (C₁-C₄)alkylsufonates or (C₁-C₄)alkylarylsulfonates.

Preference is given, among the quaternary ammonium salts of formula (I), on the one hand, to tetraalkylammonium chlorides, such as, for example, dialkyldimethylammonium or alkyltrimethylammonium chlorides, in which the alkyl group comprises approximately from 12 to 22 carbon atoms, in particular behenyltrimethylammonium, distearyldimethylammonium, cetyltrimethylammonium or benzyldimethylstearylammonium chlorides, or else palmitylamidopropyltrimethylammonium chloride or stearamidopropyldimethyl(myristyl acetate)ammonium chloride, sold under the name Ceraphyl® 70 by Van Dyk.
quaternary ammonium salts of imidazoline, such as, for example, those of the following formula (II):

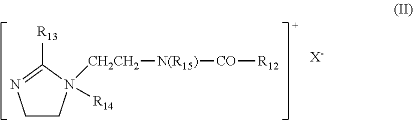

in which

R₁₂ represents an alkenyl or alkyl group comprising from 8 to 30 carbon atoms, for example derived from tallow fatty acids,

R₁₃ represents a hydrogen atom, a C₁-C₄ alkyl group or an alkenyl or alkyl group comprising from 8 to 30 carbon atoms,

R₁₄ represents a C₁-C₄ alkyl group,

R₁₅ represents a hydrogen atom or a C₁-C₄ alkyl group; X⁻ is an anion chosen from the group of halides such as chloride, bromide and iodide, phosphates, acetates, lactates, (C₁-C₄)alkyl sulfates, and (C₁-C₄)alkyl- or (C₁-C₄)alkylarylsulfonates.

Preferably, R₁₂ and R₁₃ denote a mixture of alkenyl or alkyl groups comprising from 12 to 21 carbon atoms, for example derived from tallow fatty acids, R₁₄ denotes a methyl group and R₁₅ denotes a hydrogen atom. Such a product is sold, for example, under the name Rewoquat® W 75 by Rewo;
quaternary diammonium or triammonium salts, in particular of formula (III) below:

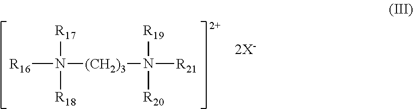

in which R₁₆ denotes an alkyl group comprising approximately from 16 to 30 carbon atoms, which is optionally hydroxylated and/or interrupted with one or more oxygen atoms,

R¹⁷ is chosen from hydrogen, an alkyl group comprising from 1 to 4 carbon atoms or a group —(CH₂)₃—N⁺(R₁₆ₐ)(R₁₇ₐ)(R₁₈ₐ),

R₁₆ₐ, R₁₇ₐ, R₁₈ₐ, R₁₈, R₁₉, R₂₀ and R₂₁, which may be identical or different, are chosen from hydrogen or an alkyl group comprising from 1 to 4 carbon atoms, and

X⁻ is an anion chosen from the group of halides, such as chloride, bromide and iodide, acetates, phosphates, nitrates, (C₁-C₄)alkyl sulfates, (C₁-C₄)alkyl- or (C₁-C₄)alkylarylsulfonates, in particular methyl sulfate and ethyl sulfate.

Such compounds are, for example, Finquat CT-P, sold by Finetex (Quaternium 89), and Finquat CT, sold by Finetex (Quaternium 75).
quaternary ammonium salts comprising one or more ester functions, for instance those of formula (IV) below:

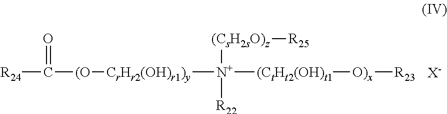

in which:

R₂₂ is chosen from C₁-C₆ alkyl groups and C₁-C₆ hydroxyalkyl or dihydroxyalkyl groups,

R₂₃ is chosen from:
the group

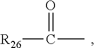

saturated or unsaturated, linear or branched C₁-C₂₂ hydrocarbon-based groups R₂₇, and
a hydrogen atom,

R₂₅ is chosen from:
the group

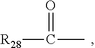

saturated or unsaturated, linear or branched C₁-C₆ hydrocarbon-based groups R₂₉, and
a hydrogen atom,

R₂₄, R₂₆ and R₂₈, which may be identical or different, are chosen from saturated or unsaturated, linear or branched C₇-C₂₁ hydrocarbon-based groups;

r, s and t, which may be identical or different, are integers having values from 2 to 6,

r1 and t1, which may be identical or different, have the values 0 or 1,

r2+r1=2 r and t1+t2=2 t,

y is an integer ranging from 1 to 10,

x and z, which may be identical or different, are integers having values from 0 to 10,

X⁻ is a simple or complex, organic or inorganic anion,

with the proviso that the sum x+y+z is from 1 to 15, that when x is 0 then R₂₃ denotes R₂₇, and that when z is 0 then R₂₅ denotes R₂₉.

The alkyl groups R₂₂ may be linear or branched, and more particularly linear.

Preferably, R₂₂ denotes a methyl, ethyl, hydroxyethyl or dihydroxypropyl group and more particularly a methyl or ethyl group.

Advantageously, the sum x+y+z has a value from 1 to 10.

When R₂₃ is a hydrocarbon-based group R₂₇, it can be long and have from 12 to 22 carbon atoms or be short and have from 1 to 3 carbon atoms.

When R₂₅ is a hydrocarbon-based group R₂₉, it preferably has from 1 to 3 carbon atoms.

Advantageously, R₂₄, R₂₆ and R_{28,} which may be identical or different, are chosen from linear or branched, saturated or unsaturated C₁₁-C₂₁ hydrocarbon-based groups, and more particularly from linear or branched, saturated or unsaturated C₁₁-C₂₁ alkyl and alkenyl groups.

Preferably, x and z, which may be identical or different, have the value 0 or 1.

Advantageously, y is equal to 1.

Preferably, r, s and t, which may be identical or different, have the value 2 or 3 and more particularly still are equal to 2.

The anion X⁻ is preferably a halide, preferably chloride, bromide or iodide, a (C₁-C₄)alkyl sulfate, or a (C₁-C₄)alkyl- or (C₁-C₄)alkylarylsulfonate. However, use may be made of methanesulfonate, phosphate, nitrate, tosylate, an anion derived from an organic acid, such as acetate or lactate, or any other anion which is compatible with the ammonium having an ester function.

The anion X⁻ is more particularly still chloride, methyl sulfate or ethyl sulfate.

Use is more particularly made, in the composition according to the invention, of the ammonium salts of formula (IV) in which:
R₂₂ denotes a methyl or ethyl group,
x and y are equal to 1,
z is equal to 0 or 1,
r, s and t are equal to 2,
R₂₃ is chosen from:
the group

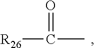

methyl, ethyl or C₁₄-C₂₂ hydrocarbon-based groups, and
a hydrogen atom,
R₂₅ is chosen from:
the group

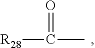

and
a hydrogen atom,
R₂₄, R₂₆ and R₂₈, which may be identical or different, are chosen from linear or branched, saturated or unsaturated C₁₃-C₁₇ hydrocarbon-based groups, and preferably from linear or branched, saturated or unsaturated C₁₃-C₁₇ alkyl and alkenyl groups.

Advantageously, the hydrocarbon-based groups are linear.

Among the compounds of formula (IV), examples that may be mentioned include salts, especially the chloride or the methyl sulfate of diacyloxyethyldimethylammonium, diacyloxyethylhydroxyethyl-methylammonium, monoacyloxyethyldihydroxyethylmethylammonium, triacyloxyethylmethylammonium or monoacyloxyethylhydroxyethyl-dimethylammonium, and mixtures thereof. The acyl groups preferably have from 14 to 18 carbon atoms and originate more particularly from a vegetable oil, such as palm oil or sunflower oil. When the compound comprises several acyl groups, the latter can be identical or different.

These products are obtained, for example, by direct esterification of triethanolamine, triisopropanolamine, alkyldiethanolamine or alkyldiisopropanolamine, which are optionally oxyalkylenated, with fatty acids or with fatty acid mixtures of plant or animal origin, or by transesterification of the methyl esters thereof. This esterification is followed by a quaternization by means of an alkylating agent such as an alkyl halide, preferably a methyl or ethyl halide, a dialkyl sulfate, preferably a dimethyl or diethyl sulfate, methyl methanesulfonate, methyl para-toluenesulfonate, glycol chlorohydrin or glycerol chlorohydrin.

Such compounds are, for example, sold under the names Dehyquart® by Henkel, Stepanquat® by Stepan, Noxamium® by Ceca or Rewoquat® WE 18 by Rewo-Witco.

The composition according to the invention can comprise, for example, a mixture of quaternary ammonium mono-, di- and triester salts with a predominance by weight of diester salts.

Mention may be made, as examples of such compounds, of distearoylethylhydroxyethylmethylammonium or dipalmitoylethylhydroxyethylmethylammonium salts, and in particular the methosulfates.

Use may also be made of the ammonium salts containing at least one ester function that are described in patents U.S. Pat. No. 4,874,554 and U.S. Pat. No. 4,137,180.

Use may also be made of behenoylhydroxypropyltrimethylammonium chloride, for example, sold by KAO under the name Quartamin BTC 131.

Preferably, the ammonium salts containing at least one ester function contain two ester functions.

Among the cationic surfactants, preference is more particularly given to those of formula (I) such as cetyltrimethylammonium salts, behenyltrimethylammonium salts, and mixtures thereof, and more particularly behenyltrimethylammonium chloride, cetyltrimethyl- ammonium chloride and mixtures thereof.

The cationic surfactant(s) is (are) preferably present in a total amount ranging from 0.01% to 20% by weight, better still from 0.1% to 10% by weight and even more preferentially still from 0.5% to 6% by weight, relative to the total weight of the composition.

Within the meaning of the present invention, the term fatty alcohol is intended to mean an alcohol comprising, in its main chain, at least one saturated or unsaturated hydrocarbon-based chain, such as alkyl or alkenyl, comprising at least 8 carbon atoms, preferably from 8 to 30 carbon atoms, and even better still from 10 to 22 carbon atoms.

They are not glycerolated or polyoxyalkylenated.

They may be saturated or unsaturated, linear or branched.

They preferably have the structure R—OH in which R denotes a linear or branched C₈₋₃₀, preferably C₁₀₋₂₂, better still C₁₂₋₂₄ alkyl or alkenyl group, R possibly being substituted by one or more hydroxyl groups.

The fatty alcohols may be liquid or non-liquid at room temperature (25° C.) and at atmospheric pressure (760 mmHg, i.e. 1.013×10⁵ Pa).

“Liquid fatty alcohol” means a non-glycerolated and non-oxyalkylenated fatty alcohol, which is liquid at room temperature (25° C.) and at atmospheric pressure (760 mmHg, i.e. 1.013×10⁵ Pa).

The liquid fatty alcohols of the invention may be saturated or unsaturated.

The saturated liquid fatty alcohols are preferably branched. They may optionally comprise in their structure at least one aromatic or non-aromatic ring. They are preferably acyclic.

The unsaturated liquid fatty alcohols contain in their structure at least one double or triple bond, and preferably one or more double bonds. When several double bonds are present, there are preferably 2 or 3 of them, and they may be conjugated or unconjugated.

These unsaturated fatty alcohols may be linear or branched.

They may optionally comprise in their structure at least one aromatic or non-aromatic ring. They are preferably acyclic.

The liquid fatty alcohols preferably have the structure R′-OH, in which R′ denotes a branched C₁₂-C₂₄ alkyl or linear or branched C₁₂-C₂₄ alkenyl group, R′ possibly being substituted by one or more hydroxyl groups.

Preferably, the liquid fatty alcohol of the invention is a branched saturated alcohol.

Preferably, R′ does not contain any hydroxyl groups.

“Non-liquid” preferably means a solid compound or a compound that has a viscosity of greater than 2 Pa·s at a temperature of 25° C. and at a shear rate of 1 s⁻¹.

The non-liquid fatty alcohols that are suitable for use in the invention are more particularly chosen from linear or branched, saturated or unsaturated alcohols comprising from 8 to 30 carbon atoms and preferably from 8 to 24 carbon atoms. Preferably, the non-liquid fatty alcohols have the structure R″-OH with R″ denoting a linear alkyl group, optionally substituted by one or more hydroxyl groups, comprising from 8 to 30, better still from 10 to 30, or even from 12 to 24 carbon atoms.

The non-liquid fatty alcohols that can be used are preferably chosen from saturated or unsaturated, linear or branched, preferably linear and saturated, (mono)alcohols comprising from 8 to 30 carbon atoms, better still from 10 to 30, or even from 12 to 24 carbon atoms.

As examples of fatty alcohols that may be used in the present invention, mention may especially be made of oleyl alcohol, linoleyl alcohol, linolenyl alcohol, undecylenyl alcohol, isocetyl alcohol, isostearyl alcohol, 2-octyl-1-dodecanol, 2-butyloctanol, 2-hexyl-1-decanol, 2-decyl-1-tetradecanol, 2-tetradecyl-1-cetanol, cetyl alcohol, stearyl alcohol, cetylstearyl alcohol, and mixtures thereof.

Preferentially, a fatty alcohol chosen from 2-octyl-1-dodecanol, oleyl alcohol, cetyl alcohol, stearyl alcohol, and mixtures thereof may be used.

More particularly, the fatty alcohols are chosen from fatty alcohols which are not liquid at room temperature (25° C.) and under atmospheric pressure (760 mmHg, i.e. 1.013×10⁵ Pa), better still from cetyl alcohol, stearyl alcohol, and mixtures thereof.

The fatty alcohol(s) are preferably present in a total amount ranging from 0.01% to 20% by weight, better still from 0.1% to 15% by weight and even more preferentially still from 1% to 10% by weight relative to the total weight of the composition.

The composition according to the invention comprises at least one nonionic non-associative cellulose-based polymer.

According to the invention, the term “cellulose-based polymer” is intended to mean any polysaccharide polymer having, in its structure, sequences of glucose residues joined together via beta-1,4 bonds.

According to the invention, the term “non-associative cellulose-based polymer” is intended to mean that the cellulose-based polymers do not comprise a C₈-C₃₀ fatty chain. The nonionic non-associative cellulose-based polymers according to the invention may be chosen from (C₁-C₄)alkylcelluloses, such as methylcelluloses and ethylcelluloses; hydroxy(C₁-C₄)alkylcelluloses, such as hydroxymethylcelluloses, hydroxyethylcelluloses and hydroxypropylcelluloses; mixed hydroxy(C₁-C₄)alkyl-(C₁-C₄)alkyl-celluloses, such as hydroxypropyl-methylcelluloses, hydroxyethyl-methylcelluloses, hydroxyethyl-ethylcelluloses, and hydroxbutyl-methylcelluloses, and also mixtures thereof.

Better still, the nonionic non-associative cellulose-based polymer(s) are chosen from hydroxy(C₁-C₄)alkylcelluloses and especially hydroxyethylcelluloses.

The nonionic non-associative cellulose-based polymer(s) used in the composition of the invention are present in a total amount preferably ranging from 0.01% to 10% by weight, better still from 0.05% to 5% by weight and even better still from 0.1% to 3% by weight relative to the total weight of the composition.

The composition according to the invention comprises at least one nonionic associative polymer.

Within the meaning of the present invention, the term “associative polymer” is intended to mean an amphiphilic polymer that is capable, in an aqueous medium, of reversibly combining with itself or with other molecules. It generally comprises, in its chemical structure, at least one hydrophilic region or group and at least one hydrophobic region or group.

The term “hydrophobic region or group” is intended to mean a hydrocarbon-based group or a polymer comprising a saturated or unsaturated, linear or branched hydrocarbon-based chain. When it denotes a hydrocarbon-based group, the hydrophobic group comprises at least 8 carbon atoms, preferably from 8 to 30 carbon atoms, in particular from 8 to 24 carbon atoms and preferentially from 10 to 24 carbon atoms. Preferentially, the hydrophobic group is derived from a monofunctional compound. By way of example, the hydrophobic group may be derived from a fatty alcohol, such as stearyl alcohol, dodecyl alcohol or decyl alcohol, or else from a polyalkylenated fatty alcohol, such as Steareth-100. It may also denote a hydrocarbon-based polymer, such as, for example, polybutadiene.

The term “fatty chain” is intended hereinafter to mean a saturated or unsaturated, linear, branched or cyclic hydrocarbon-based chain, preferably alkyl, comprising at least 8 carbon atoms, preferably from 8 to 30 carbon atoms, in particular from 8 to 24 carbon atoms and preferentially from 10 to 24 carbon atoms.

The non-ionic associative polymers which can be used in the invention are preferably chosen from:

(1) celluloses modified by groups comprising at least one fatty chain. Mention may be made, by way of example, of:
hydroxyethylcelluloses modified by groups comprising at least one fatty chain, such as alkyl, arylalkyl or alkylaryl groups, or mixtures thereof, and in which the alkyl groups are preferably C₈-C₂₂, such as the products Natrosol Plus Grade 330 CS (C16 alkyls) or Polysurf 67 CS sold by Ashland, or the product Bermocoll EHM 100 sold by Berol Nobel,
hydroxyethylcelluloses modified by polyalkylene glycol (C₈-C₂₂ alkyl)phenyl ether groups, such as the product Amercell Polymer HM-1500 (nonylphenyl polyethylene glycol (15) ether) sold by Amerchol;

(2) hydroxypropyl guars modified by groups comprising at least one fatty chain, such as the product Esaflor HM 22 (C₂₂ alkyl chain), sold by Lamberti, and the products RE210-18 (C₁₄ alkyl chain) and RE205-1 (C₂₀ alkyl chain), sold by Rhodia Chimie;

(3) inulins modified by groups comprising at least one fatty chain, such as inulin alkyl carbamates and in particular the inulin lauryl carbamate provided by Orafti under the name Inutec SPI;

(4) copolymers of vinylpyrrolidone and of fatty-chain hydrophobic monomers, of which mention may be made, by way of example, of:
the products Antaron V216 or Ganex V216 (vinylpyrrolidone/hexadecene copolymer), sold by ISP,
the products Antaron V220 or Ganex V220 (vinylpyrrolidone/eicosene copolymer), sold by ISP;

(5) copolymers of C₁-C₆ alkyl methacrylates or acrylates and of amphiphilic monomers comprising at least one fatty chain, such as, for example, the methyl acrylate/oxyethylenated stearyl acrylate copolymer sold by Goldschmidt under the name Antil 208;

(6) copolymers of hydrophilic methacrylates or acrylates and of hydrophobic monomers comprising at least one fatty chain, such as, for example, the polyethylene glycol methacrylate/lauryl methacrylate copolymer;

(7) polyurethane polyethers comprising, in their chain, both hydrophilic blocks usually of polyoxyethylenated nature and hydrophobic blocks which can be aliphatic sequences alone and/or cycloaliphatic and/or aromatic sequences;

(8) polymers comprising an aminoplast ether backbone having at least one fatty chain, such as the Pure Thix compounds offered by Sü-Chemie.

The nonionic associative polymer(s) are preferably chosen from celluloses modified by groups comprising at least one fatty chain of family (1), in particular hydroxyethylcelluloses modified by groups comprising at least one fatty chain such as alkyl, arylalkyl or alkylaryl groups or mixtures thereof, and in which the alkyl groups are preferably C8⁻C22, and polyurethane polyethers of family (7). Preferentially, the nonionic associative polymer(s) are chosen from the polyurethane polyethers of family (7).

Preferably, the nonionic associative polyurethane polyethers according to the invention comprise at least two lipophilic hydrocarbon-based chains having from 8 to 30 carbon atoms which are separated by a hydrophilic block, it being possible for the hydrocarbon-based chains to be pendent chains or chains at the end of the hydrophilic block. In particular, it is possible for one or more pendent chains to be envisaged. In addition, the polymer may comprise a hydrocarbon-based chain at one end or at both ends of a hydrophilic block.

The polyurethane polyethers can be multiblock, in particular in triblock form. The hydrophobic blocks can be at each end of the chain (for example: triblock copolymer having a hydrophilic central block) or distributed both at the ends and in the chain (for example, multiblock copolymer). These same polymers can also be graft polymers or star polymers.

The nonionic fatty-chain polyurethane polyethers can be triblock copolymers, the hydrophilic block of which is a polyoxyethylene chain comprising from 50 to 1000 oxyethylene groups. The nonionic polyurethane polyethers comprise a urethane bond between the hydrophilic blocks, hence the origin of the name.

By extension, the nonionic fatty-chain polyurethane polyethers also include those with hydrophilic blocks bonded to the lipophilic blocks via other chemical bonds.

Mention may also be made, as examples of nonionic fatty-chain polyurethane polyethers which can be used in the invention, of Rheolate 205 comprising a urea function, sold by Elementis, or else Rheolate 208, 204 or 212, and also Acrysol RM 184.

Mention may also be made of the product Elfacos T210 containing a C₁₂-C₁₄ alkyl chain, and the product Elfacos T212 containing a C₁₈ alkyl chain, from Akzo.

The product DW 1206B from Rohm & Haas having a C₂₀ alkyl chain and a urethane bond, provided at a solids content of 20% in water, may also be used.

Use may also be made of solutions or dispersions of these polymers, especially in water or in aqueous/alcoholic medium. Mention may be made, as examples of such polymers, of Rheolate 255, Rheolate 278 and Rheolate 244, sold by Elementis. Use may also be made of the products DW 1206F and DW 1206J sold by Rohm & Haas.

The polyurethane polyethers which can be used according to the invention are in particular those described in the paper by G. Fonnum, J. Bakke and Fk. Hansen—Colloid Polym. Sci 271, 380.389 (1993).

In one variant, use is made of a nonionic associative polyurethane polyether able to be obtained by polycondensation of at least three compounds comprising (i) at least one polyethylene glycol comprising from 100 to 180 mol of ethylene oxide, (ii) a polyoxyethylenated stearyl alcohol comprising 100 mol of ethylene oxide and (iii) a diisocyanate.

Such a polymer is especially sold by Elementis under the name Rheolate FX 1100®, which is a polycondensate of polyethylene glycol containing 136 mol of ethylene oxide, of polyoxyethylenated stearyl alcohol with 100 mol of ethylene oxide and of hexamethylene diisocyanate (HDI) with a weight-average molecular weight (Mw) of 30 000 (INCI name: PEG-136/Steareth-100/HDI Copolymer).

According to another variant, use may also be made of a polyurethane polyether able to be obtained by polycondensation of at least three compounds comprising (i) at least one polyethylene glycol comprising from 150 to 180 mol of ethylene oxide, (ii) stearyl alcohol or decyl alcohol and (iii) at least one diisocyanate.

Such polyurethane polyethers are sold especially by Röhm & Haas under the names Aculyn 46 and Aculyn 44 [Aculyn 46 is a polycondensate of polyethylene glycol comprising 150 or 180 mol of ethylene oxide, of stearyl alcohol and of methylenebis(4-cyclohexyl isocyanate) (SMDI), at 15% by weight in a matrix of maltodextrin (4%) and water (81%); Aculyn 44 is a polycondensate of polyethylene glycol (PEG) comprising 150 or 180 mol of ethylene oxide, of decyl alcohol and of methylenebis(4-cyclohexyl isocyanate) (SMDI), at 35% by weight in a mixture of propylene glycol (39%) and water (26%)].

Preferably, the associative polymers as described above have a number-average molecular weight of less than 500 000 and even more preferentially of less than 100 000, preferably ranging from 5000 to 80 000, which can be measured by methods such as cryoscopy, osmotic pressure, ebullioscopy or titration of the end groups.

Preferentially, the nonionic associative polymer used in the composition according to the invention is a nonionic associative polymer chosen from the nonionic associative polyurethane polyethers (family (7)), in particular those able to be obtained by polycondensation of at least three compounds comprising (i) at least one polyethylene glycol comprising from 150 to 180 mol of ethylene oxide, (ii) stearyl alcohol or decyl alcohol and (iii) at least one diisocyanate.

Even more particularly, the nonionic associative polymer(s) used in the invention is (are) chosen from the polycondensate of polyethylene glycol comprising 150 or 180 mol of ethylene oxide, of decyl alcohol and of methylenebis(4-cyclohexyl isocyanate) (SMDI) sold under the name ACULYN 44, or the polycondensate of polyethylene glycol comprising 150 or 180 mol of ethylene oxide, of stearyl alcohol and of methylenebis(4-cyclohexyl isocyanate) (SMDI) sold under the name ACULYN 46, and mixtures thereof.

The nonionic associative polymer(s) used in the composition of the invention are present in a total amount preferably ranging from 0.01% to 10% by weight, better still from 0.02% to 5% by weight and even more preferentially ranging from 0.05% to 2% by weight relative to the total weight of the composition.

In the composition according to the invention, the weight ratio between the total amount of nonionic non-associative cellulose-based polymer(s) and the total amount of nonionic associative polymer(s) ranges from 1 to 20. Preferably, this weight ratio ranges from 1 to 15, better still from 1.5 to 10 and even more preferentially from 2 to 5.

The composition according to the invention may also comprise one or more silicones, preferably amino silicones.

The term “amino silicone” is intended to mean any silicone comprising at least one primary, secondary or tertiary amine function or a quaternary ammonium group.

Preferably, the amino silicone(s) used in the cosmetic composition according to the present invention comprise in their structure at least 4 silicon atoms.

Throughout the text hereinbelow, the term “silicone” is intended to denote, in accordance with what is generally accepted, any organosilicon polymers or oligomers of linear or cyclic, branched or crosslinked structure, of variable molecular weight, obtained by polymerization and/or polycondensation of suitably functionalized silanes, and constituted essentially of a repetition of main units in which the silicon atoms are linked together via oxygen atoms (siloxane bond —Si—O—Si—), optionally substituted hydrocarbon-based groups being directly linked via a carbon atom to said silicon atoms. The hydrocarbon-based groups that are the most common are alkyl groups, especially C₁-C₁₀ and in particular methyl, fluoroalkyl groups, the alkyl part of which is C₁-C₁₀, and aryl groups and in particular phenyl.

The amino silicones used in the composition according to the present invention may be chosen from:

(a) the compounds corresponding to the following formula (V):

(R¹)ₐ(T)₃₋ₐ—Si[OSi(T)₂]ₙ—[OSi(T)_{b}(R¹)_{2-b}]ₘ—OSi(T)₃₋ₐ—(R¹)ₐ (V)

in which:

T is a hydrogen atom or a phenyl or hydroxyl (—OH), or C₁-C₈ alkyl, and preferably methyl, or C₁-C₈ alkoxy, preferably methoxy, group,

a denotes the number 0 or an integer from 1 to 3, and preferably 0,

b denotes 0 or 1, and in particular 1,

m and n are numbers such that the sum (n+m) can range especially from 1 to 2000 and in particular from 50 to 150, it being possible for n to denote a number from 0 to 1999 and especially from 49 to 149, and for m to denote a number from 1 to 2000 and especially from 1 to 10,

R¹ is a monovalent group of formula —C_{q}H_{2q}L in which q is a number from 2 to 8 and L is an optionally quaternized amino group chosen from the following groups:
—(R²)—CH₂—CH₂—N(R²)₂,
—N(R²)₂,
—N⁺(R²)₃ Q⁻,
—N⁺(R²) (H)₂ Q⁻,
—N⁺(R²)₂HQ⁻,
—N(R²)—CH₂—CH₂—N⁺(R²)(H)₂Q⁻,
   in which R² may denote a hydrogen atom, a phenyl, benzyl or saturated monovalent hydrocarbon-based group, for example a C₁-C₂₀ alkyl group, and Q⁻ represents a halide ion, for example fluoride, chloride, bromide or iodide.

In particular, the amino silicones corresponding to the definition of formula (V) are chosen from the compounds corresponding to the following formula (VI):

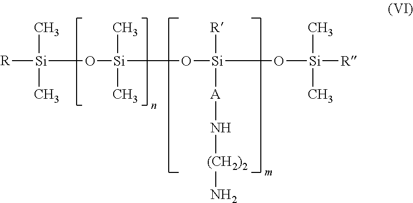

in which R, R′ and R″, which may be identical or different, denote a C₁-C₄ alkyl group, preferably CH₃; a C₁-C₄ alkoxy group, preferably methoxy; or OH; A represents a linear or branched, C₃-C₈ and preferably C₃-C₆ alkylene group; m and n are integers dependent on the molecular weight and the sum of which is between 1 and 2000.

According to a first possibility, R, R′ and R″, which may be identical or different, each represent a C₁-C₄ alkyl group or hydroxyl, A represents a C₃ alkylene group and m and n are such that the weight-average molecular weight of the compound is between approximately 5000 and 500 000. Compounds of this type are referred to in the CTFA dictionary as “amodimethicones”.

According to a second possibility, R, R′ and R″, which may be identical or different, each represent a C₁-C₄ alkoxy group or hydroxyl, at least one of the groups R and R″ is an alkoxy group and A represents a C₃ alkylene group. The hydroxyl/alkoxy mole ratio is preferably between 0.2/1 and 0.4/1 and advantageously equal to 0.3/1. Moreover, m and n are such that the weight-average molecular weight of the compound is between 2000 and 10⁶. More particularly, n is between 0 and 999 and m is between 1 and 1000, the sum of n and m being between 1 and 1000.

In this category of compounds, mention may be made, inter alia, of the product Belsil® ADM 652 sold by Wacker.

According to a third possibility, R and R″, which are different, each represent a C₁-C₄ alkoxy group or hydroxyl, at least one of the groups R and R″ being an alkoxy group, R′ representing a methyl group and A representing a C₃ alkylene group. The hydroxyl/alkoxy mole ratio is preferably between 1/0.8 and 1/1.1 and is advantageously equal to 1/0.95. Moreover, m and n are such that the weight-average molecular weight of the compound is between 2000 and 200 000. More particularly, n is between 0 and 999 and m is between 1 and 1000, the sum of n and m being between 1 and 1000.

More particularly, mention may be made of the product Fluid WR® 1300 sold by Wacker.

Note that the molecular weight of these silicones is determined by gel permeation chromatography (room temperature, polystyrene standard, μ styragem columns, eluent THF, flow rate of 1 mm/minute, 200 μl of a solution containing 0.5% by weight of silicone in THF are injected, and detection is performed by refractometry and UV-metry).

A product corresponding to the definition of formula (V) is in particular the polymer known in the CTFA dictionary as Trimethylsilyl Amodimethicone, corresponding to formula (VII) below:

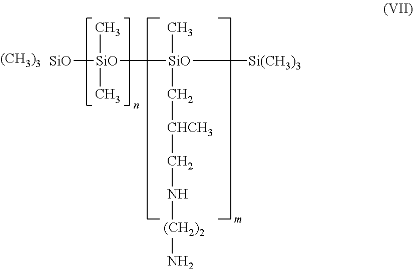

in which n and m have the meanings given above in accordance with formula (V).

Such compounds are described, for example, in EP 95238. A compound of formula (V) is sold, for example, under the name Q2-8220 by OSI.

(b) the compounds corresponding to formula (VIII) below:

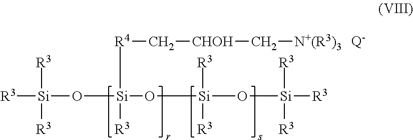

in which:

R³ represents a monovalent C₁-C₁₈ hydrocarbon-based group, and in particular a C₁-C₁₈ alkyl or C₂-C₁₈ alkenyl group, for example methyl,

R⁴ represents a divalent hydrocarbon-based group, especially a C₁-C₁₈ alkylene group or a divalent C₁-C₁₈, for example C₁-C₈, alkyleneoxy group,

Q⁻ is a halide ion, especially chloride,

r represents a mean statistical value from 2 to 20 and in particular from 2 to 8,

s represents a mean statistical value from 20 to 200 and in particular from 20 to 50.

Such compounds are described more particularly in patent U.S. Pat. No. 4,185,087.

A compound falling within this class is the product sold by Union Carbide under the name Ucar Silicone ALE 56.

(c) quaternary ammonium silicones, especially of formula (IX):

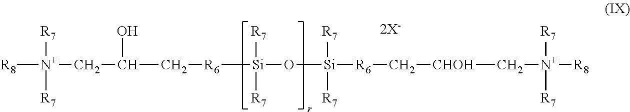

in which:

R₇, which may be identical or different, represent a monovalent hydrocarbon-based group having from 1 to 18 carbon atoms, and in particular a C₁-C₁₈ alkyl group, a C₂-C₁₈ alkenyl group or a ring comprising 5 or 6 carbon atoms, for example methyl,

R₆ represents a divalent hydrocarbon-based group, especially a C₁-C₁₈ alkylene group or a divalent C₁-C_{18,} for example C₁-C₈, alkyleneoxy group linked to the Si via an Si—C bond,

R₈, which may be identical or different, each represent a hydrogen atom, a monovalent hydrocarbon-based group containing from 1 to 18 carbon atoms, and in particular a C₁-C₁₈ alkyl group, a C₂-C₁₈ alkenyl group or an —R₆—NHCOR₇ group;

X⁻ is an anion, such as a halide ion, especially chloride, or an organic acid salt (acetate, etc.),

r represents a mean statistical value from 2 to 200 and in particular from 5 to 100.

These silicones are described, for example, in application EP-A 0 530 974.

(d) the amino silicones of formula (X):

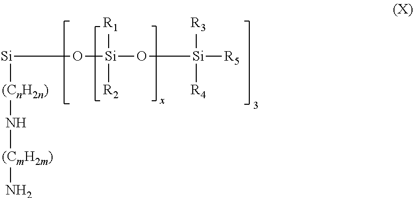

in which:
R₁, R₂, R₃ and R₄, which may be identical or different, each denote a C₁-C₄ alkyl group or a phenyl group,
R5 denotes a C₁-C₄ alkyl group or a hydroxyl group,
n is an integer ranging from 1 to 5,
m is an integer ranging from 1 to 5, and
x is chosen such that the amine number is between 0.01 and 1 meq/g.

When these compounds are used, one particularly advantageous embodiment involves their combined use with cationic and/or nonionic surfactants.

By way of example, use may be made of the product sold under the name Cationic Emulsion DC939 by Dow Corning, which comprises, aside from amodimethicone, a cationic surfactant, namely trimethylcetylammonium chloride and a nonionic surfactant of formula C₁₃H₂₇—(OC₂H₄)₁₂—OH, known under the CTFA name Trideceth-12.

Another commercial product that may be used according to the invention is the product sold under the name Dow Corning Q2 7224 by Dow Corning, comprising, in combination, the trimethylsilyl amodimethicone of formula (VII) described above, a nonionic surfactant of formula: C₈H₁₇—C₆H₄—(OCH₂CH₂)₄₀—OH, known under the CTFA name Octoxynol-40, a second nonionic surfactant of formula: C₁₂H₂₅—(OCH₂—CH₂)₆—OH, known under the CTFA name Isolaureth-6, and propylene glycol.

Another commercial product that may be used according to the invention is the product sold under the name Wacker-Belsil ADM LOG 1, sold by Wacker, comprising, in microemulsion form, an amodimethicone of formula (VI) in combination with Trideceth-5 and Trideceth-10.

It is also possible to use the product sold under the trade name Xiameter MEM 8299 emulsion by Dow Corning.

Other amino silicones may be used within the context of the invention, such as the product which is referenced in the CTFA dictionary under the name Polysilicone-9.

Preferably, the amino silicone(s) used in the cosmetic composition according to the invention are chosen from the amino silicones corresponding to formula (V), and even more particularly from the amino silicones of formulae (VI) or (VII).

Preferably, the composition according to the invention comprises an amino silicone, better still a non-phenyl amino silicone.

When the amino silicone(s) are present in the composition according to the invention, the amount thereof preferably ranges from 0.01% to 10% by weight, better still from 0.1% to 5% by weight, and even more preferentially from 0.5% to 3% by weight, relative to the total weight of the composition.

The compositions according to the invention may also contain one or more additives chosen from cationic, anionic, amphoteric or zwitterionic polymers, non-ionic polymers other than those set out above, non-ionic, anionic, amphoteric or zwitterionic surfactants, ceramides, pseudoceramides, vitamins and provitamins including panthenol, water-soluble and liposoluble sunscreens, nacreous agents, opacifiers, sequestrants, solubilizers, antioxidants, antidandruff agents, anti-seborrheic agents, agents for preventing hair loss and/or for promoting hair growth, penetrants, fragrances, peptizing agents and preservatives, or any other additive conventionally used in the cosmetics field.

These additives may be present in the composition according to the invention in an amount ranging from 0 to 20% by weight relative to the total weight of the composition.

Those skilled in the art will take care to select these optional additives and the amounts thereof so that they do not harm the properties of the compositions of the present invention.

The compositions according to the invention may, in a non-limiting manner, be in the form of care products to be applied, where appropriate, before and/or after shampooing, or dyeing, bleaching, permanent-waving, relaxing or styling products.

The present invention also relates to a process for cosmetic treatment, and more particularly for conditioning, of keratin materials, especially keratin fibres and especially the hair, which comprises the application, to said materials, of an effective amount of a composition as described above.

This application may or may not be followed by a rinsing operation.

When the application of the composition is followed by a rinsing operation, the leave-in time of the composition on the keratin materials ranges from a few seconds to 60 minutes, better still from 5 seconds to 30 minutes and even better still from 10 seconds to 10 minutes.

Whether in rinse-out mode or leave-in mode, the application of the composition may take place in the presence or absence of heat. The heating device may be a hairdryer, a hood dryer, a curling iron or a flat iron. The heating temperature may be between 40° C. and 220° C.

The present invention also relates to the use of the cosmetic composition according to the invention for conditioning the hair, or as hair care products. As examples of care products, mention may especially be made of conditioners, masks, and leave-in care products.

The invention is illustrated by the following examples.

### EXAMPLES

### Example 1

The composition according to the invention was prepared from the ingredients indicated in the table below. The amounts indicated are expressed in % by weight of active substance relative to the total weight of the composition.

| | |
|---|---|
| Cetearyl alcohol | 4% |
| (Nafol 1618 EN from Sasol) | |
| Behenyltrimethylammonium chloride | 2% |
| (Genamin KDMP from Clariant) | |
| Amodimethicone (Xiameter MEM-8299 | 1% |
| Emulsion from Dow Corning) | |
| Hydroxyethylcellulose | 0.3% |
| (Natrosol 250 HHR CS from Ashland) | |
| PEG-150/decyl alcohol/SMDI copolymer | 0.1% |
| (Aculyn 44 from Dow Chemical) | |
| Water | qs 100% |

After preparation of the composition, it was applied to hair.

### Example 2

The following compositions A and B were prepared from the ingredients indicated in the table below. The amounts are indicated in % by weight of active substance relative to the total weight of the composition.

| | A | B |
|---|---|---|
| INCI Name | (invention) | (comparative) |
| Cetearyl alcohol | 4 | 4 |
| Behentrimonium chloride | 1.8 | 1.8 |
| Amodimethicone | 1 | 1 |
| Hydroxyethylcellulose | 0.3 | — |
| Cetyl hydroxyethylcellulose | — | 0.3 |
| PEG-150/Decyl | 0.2 | 0.2 |
| Alcohol/SMDI Copolymer | | |
| Citric acid | qs pH 3.2 | qs pH 3.2 |
| Water | qs 100 | qs 100 |
| Behentrimonium chloride | 240 | 220 |
| deposit (μg/g of hair) | | |

1.08 g of composition was applied to 2.7 g locks of sensitized hair, washed beforehand with DOP shampoo. 3 locks were produced for each composition.

The deposit of behentrimonium chloride on the hair locks was quantified by desorption followed by gas chromatography method (GC) combined with a flame ionization detector (FID).

Specifically, each treated hair lock was immersed in a dichloromethane solution for three hours under stirring.

The liquid extract obtained was evaporated and the final extract thus-obtained was put in a mixture of methanol and nonanol and analysed by GC/FID.

The quantification of Behentrimonium chloride in the liquid extract was carried out with an external standardization prepared in the uptake solution (methanol) and corrected by the answer of a standard (nonanol).

Then, it was observed that the deposits of Behentrimonium chloride were significantly higher with the formulation according to the invention.

Moreover, applications to locks showed a greater “on surface” effect with composition A. Better distribution of composition A, and rapid disappearance of this composition after application, was obtained compared to composition B.

1. Cosmetic composition comprising:
(a) one or more cationic surfactants,
(b) one or more fatty alcohols,
(c) one or more nonionic non-associative cellulose-based polymers, and
(d) one or more nonionic associative polymers,
the weight ratio between the total amount of nonionic non-associative cellulose-based polymer(s) and that of nonionic associative polymer(s) ranging from 1 to 20.

2. Composition according to claim 1, characterized in that the cationic surfactant(s) is (are) chosen from those corresponding to the following general formula (I): 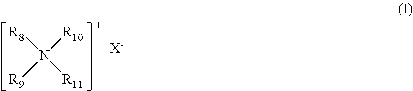
in which the groups R₈ to R₁₁, which may be identical or different, represent a linear or branched aliphatic group comprising from 1 to 30 carbon atoms, or an aromatic group such as aryl or alkylaryl, at least one of the groups R₈ to R₁₁ comprising from 8 to 30 carbon atoms and preferably from 12 to 24 carbon atoms; and X⁻ is an anion chosen from the group of halides such as chloride, bromide and iodide, phosphates, acetates, lactates, (C₁-C₄)alkyl sulfates, and (C₁-C₄)alkyl- or (C₁-C₄)alkylarylsulfonates, preferably from cetyltrimethylammonium salts, behenyltrimethylammonium salts, and mixtures thereof, and more particularly from behenyltrimethylammonium chloride, cetyltrimethylammonium chloride and mixtures thereof.

3. Composition according to claim 1 or 2, characterized in that the cationic surfactant(s) is (are) present in a total amount ranging from 0.01% to 20% by weight, preferably from 0.1% to 10% by weight and better still from 0.5% to 6% by weight, relative to the total weight of the composition.

4. Composition according to any one of the preceding claims, characterized in that the fatty alcohol(s) are chosen from alcohols that are not liquid at 25° C. and under atmospheric pressure (1.013×10⁵ Pa), better still from cetyl alcohol, stearyl alcohol, and mixtures thereof.

5. Composition according to any one of the preceding claims, characterized in that the fatty alcohol(s) are present in a total amount ranging from 0.01% to 20% by weight, and preferably from 0.1% to 15% by weight and better still from 1% to 10% by weight relative to the total weight of the composition.

6. Composition according to any one of the preceding claims, characterized in that the nonionic non-associative cellulose-based polymer(s) are chosen from hydroxy(C₁-C₄)alkylcelluloses and especially hydroxyethylcelluloses.

7. Composition according to any one of the preceding claims, characterized in that the nonionic non-associative cellulose-based polymer(s) are present in a total amount ranging from 0.01% to 10% by weight, and preferably from 0.05% to 5% by weight and better still from 0.1% to 3% by weight relative to the total weight of the composition.

8. Composition according to any one of the preceding claims, characterized in that the nonionic associative polymer(s) are chosen from celluloses modified by groups comprising at least one fatty chain, in particular hydroxyethylcelluloses modified by groups comprising at least one fatty chain such as alkyl, arylalkyl or alkylaryl groups or mixtures thereof, and in which the alkyl groups are preferably C₈-C₂₂, and polyurethane polyethers.

9. Composition according to any one of the preceding claims, characterized in that the nonionic associative polymer(s) is (are) chosen from polyurethane polyethers, preferably from nonionic associative polyurethane polyethers able to be obtained by polycondensation of at least three compounds comprising (i) at least one polyethylene glycol comprising from 150 to 180 mol of ethylene oxide, (ii) stearyl alcohol or decyl alcohol and (iii) at least one diisocyanate.

10. Composition according to any one of the preceding claims, characterized in that the nonionic associative polymer(s) are present in a total amount ranging from 0.01% to 10% by weight, and preferably from 0.02% to 5% by weight and better still from 0.05% to 2% by weight relative to the total weight of the composition.

11. Composition according to any one of the preceding claims, characterized in that the weight ratio between the total amount of nonionic non-associative cellulose-based polymer(s) and that of nonionic associative polymer(s) ranges from 1 to 15, better still from 1.5 to 10 and even more preferentially from 2 to 5.

12. Composition according to any one of the preceding claims, characterized in that it comprises one or more amino silicones.

13. Composition according to claim 12, characterized in that the amino silicone is present in an amount ranging from 0.01% to 10% by weight, preferably from 0.1% to 5% by weight and better still from 0.5% to 3% by weight, relative to the total weight of the composition.

14. Process for cosmetic treatment and more particularly for conditioning of keratin materials, in particular human keratin fibres, especially the hair, comprising the application, to said materials, of an effective amount of a composition according to any one of the preceding claims.

15. Use of the cosmetic composition according to any one of claims 1 to 13 for the cosmetic treatment of keratin materials, in particular human keratin fibres, and more particularly human hair, and most particularly for conditioning the hair, or as hair care products.
